(19) United States
(12) Patent Application Publication
(10) Pub. No.: US 2025/0381522 A1
(43) Pub. Date: Dec. 18, 2025

(54) SYSTEM FOR SYNERGISTIC AIR DETOXIFICATION AND FOOD PRODUCTION

(71) Applicants: Pierre Herckes, Gilbert, AZ (US); Paul K. Westerhoff, Scottsdale, AZ (US); Ferran Garcia-Pichel, Phoenix, AZ (US)

(72) Inventors: Pierre Herckes, Gilbert, AZ (US); Paul K. Westerhoff, Scottsdale, AZ (US); Ferran Garcia-Pichel, Phoenix, AZ (US)

(21) Appl. No.: 19/238,294

(22) Filed: Jun. 13, 2025

Related U.S. Application Data
(60) Provisional application No. 63/661,243, filed on Jun. 18, 2024.

(51) Int. Cl. B01D 53/86; B01D 53/88; C12N 1/20

(52) U.S. Cl. CPC B01D 53/8671; B01D 53/8693; B01D 53/885; C12N 1/20; B01D 2255/802; B01D 2257/504; B01D 2258/06

(57) ABSTRACT

Producing biomass includes providing a gas including carbon dioxide to a reaction chamber including a reaction medium, photocatalytically converting the carbon dioxide in the reaction medium into organic carbon-containing compounds, growing the bacteria in the reaction medium, and harvesting the bacteria. The reaction medium is an aqueous mixture including bacteria and trace elements. Photocatalytically converting carbon dioxide includes providing light to side-emitting optical fibers positioned in the reaction medium. An air purification system includes a single reaction chamber configured to contain bacteria, first and second inlets, one or more side-emitting optical fibers, and an outlet configured to allow harvesting of the bacteria. Producing bacterial biomass and purifying air includes providing a gaseous feedstock to a reaction chamber including a reaction medium, photocatalytically converting carbon dioxide into organic carbon-containing compounds, growing the bacteria in the reaction medium, harvesting the bacteria, and removing a gaseous output stream from the reaction chamber.

### CROSS-REFERENCE TO RELATED APPLICATION

This application claims the benefit of U.S. Patent Application No. 63/661,243 filed on Jun. 18, 2024, which is incorporated by reference herein in its entirety.

### TECHNICAL FIELD

This invention relates to systems for synergistic air detoxification and food production.

### BACKGROUND

Global challenges such as climate change, driven by excess carbon dioxide (CO₂) in the atmosphere, contribute to resource depletion including food and water shortages. Technologies developed to mitigate CO₂ levels include reforestation and afforestation, which absorb CO₂ through photosynthesis. Carbon capture and storage technologies capture CO₂ emissions from industrial sources and store the CO₂ underground. Direct air capture systems extract CO₂ from the air for subsequent use or sequestration. Methods such as desalination, efficient water recycling systems, and cloud seeding have been developed to provide clean and safe drinking water or increase rain fall. Approaches to address food shortages, include the development of drought-resistant crops, vertical farming techniques, and food preservation methods to reduce waste.

### SUMMARY

This disclosure describes a system (e.g., a bioreactor) that uses light (sunlight or artificial light), atmospheric gases (e.g., CO₂ and nitrogen oxides (NO_{X})), and bacteria plus trace metals or nutrients (e.g., iron, phosphorus) in atmospheric dust to produce food (e.g., biomass, including protein, lipids, carbohydrates, and antioxidants), while decontaminating the air. Bacteria harvested from the atmosphere can grow in liquid suspensions with atmospheric components (e.g., NO_{X}, dust, CO₂, single carbon organic compounds) as nutrition sources. Bacteria native to life cycles in the air can survive and metabolically outcompete other microorganisms for these atmospheric components. The bacteria can metabolize carbon, nitrogen, and phosphorus-containing compounds, as well as essential trace elements found in atmospheric components, detoxifying the air in the process.

Light delivered into the lumen of optical fibers coated with photocatalysts in the system transforms CO₂ into organic C₁ compounds such as formic acid. Air flowing through the system provides bacterial inoculants for converting the organic C₁ compounds to cellular biomass. The photocatalysis process can occur in water under conditions suitable for growth of microorganisms present in the atmosphere.

Systems can be operated using atmospheric components as feedstock. Atmospheric bacteria can convert C₁ species such as formaldehyde into biomass or food over a range of conditions (e.g., pH, temperature, ionic strength, light). The bacteria can be combined in a single reaction chamber with photocatalytically generated C₁ species, enabling air purification through the removal of C₁ contaminants such as formaldehyde, methanol, and formic acid, as well as reducing the concentration of CO₂ in the air.

In a first general aspect, producing biomass includes providing a gas to a reaction chamber including a reaction medium, photocatalytically converting the carbon dioxide in the reaction medium into organic carbon-containing compounds, growing the bacteria in the reaction medium, and harvesting the bacteria. The gas includes carbon dioxide. The reaction medium is an aqueous mixture including bacteria and a source of trace elements for growth of the bacteria. Photocatalytically converting the carbon dioxide includes providing light to side-emitting optical fibers coated on their exterior surface with a photocatalyst and positioned in the reaction medium.

Implementations of the first general aspect can include one or more of the following features.

In some cases, the gas includes air. The air can include the trace elements. Suitable examples of the trace elements include nitrogen and phosphorous. In some implementations, the gas includes particulate matter, and the particulate matter is the source of the trace elements. The particulate matter can include dust. In some examples, the source of the trace elements essential for biological growth includes dust, aerosols, or other particulates from the air.

The first general aspect can further include providing the dust, aerosols, or other particulates to the reaction chamber. Providing the dust to the reaction chamber can include filtering the dust, aerosols, or other particulates from air. In some cases, the bacteria include Proteobacteria, uncultured Caulobacteraceae, Bacteroidota, Methylobacteria, Massilia, Sphingomonas, Bacteroidetes, Firmicutes, Actinobacteria, Cyanobacteria, or a combination thereof.

In a second general aspect, an air purification system includes a single reaction chamber configured to contain bacteria, a first inlet configured to accept a first feedstock including carbon dioxide, a second inlet configured to accept a second feedstock including particulate matter from ambient air, one or more side-emitting optical fibers configured to be positioned in the single reaction chamber, and an outlet configured to allow harvesting of the bacteria from the single reaction chamber. The one or more side-emitting optical fibers include a photocatalytic exterior coating and are configured to be optically coupled to a light source and convert the carbon dioxide in the single reaction chamber to organic carbon compounds,

Implementations of the second general aspect can include one or more of the following features.

In some cases, the first feedstock is air, and the first inlet is configured to accept the air and provide the air to an aqueous reaction medium in the single reaction chamber. In some implementations, the first inlet and the second inlet are the same, the first feedstock and the second feedstock are the same, and the first feedstock and the second feedstock is air. The second general aspect can further include a system configured to capture particulate matter from ambient air and provide the particulate matter to the single reaction chamber. The system can be configured to be operatively coupled to the single reaction chamber. In some cases, the system includes a filter. The filter can be configured to capture the particulate matter from the ambient air.

In a third general aspect, producing bacterial biomass and purifying air includes providing a gaseous feedstock to a reaction chamber including a reaction medium, photocatalytically converting, in the reaction medium, carbon dioxide from the air into organic carbon-containing compounds, growing the bacteria in the reaction medium, harvesting the bacteria, and removing a gaseous output stream from the reaction chamber. The reaction medium includes bacteria, and the gaseous feedstock includes air. Photocatalytically converting the carbon dioxide includes providing light to side-emitting optical fibers coated on their exterior surface with a photocatalyst and positioned in the reaction medium. The organic carbon-containing compounds and trace elements from the air provide a source of nutrients for the bacteria. The gaseous output stream includes the air from which the carbon dioxide and the trace elements have been removed.

Implementations of the third general aspect may include one or more of the following features.

The third general aspect may further include processing the bacteria to yield foodstuff. In some implementations, the third general aspect further includes removing particulate matter from the air prior to providing the gaseous feedstock to the reaction chamber. The bacteria can be provided to the reaction chamber via particulate matter in the air. In some examples, the gaseous output stream includes the air from which the particulate matter has been removed.

The applications of a synergistic air detoxification and food production technology are numerous, ranging from producing food from the air in a single reaction chamber to combining this technology with CO₂ capture devices for CO₂ valorization and the remediation of industrial gas emissions at the source.

The details of one or more embodiments of the subject matter of this disclosure are set forth in the accompanying drawings and the description. Other features, aspects, and advantages of the subject matter will become apparent from the description, the drawings, and the claims.

### BRIEF DESCRIPTION OF DRAWINGS

FIG. 1 is a flow chart showing operations in a process for producing biomass.

FIG. 2 depicts an exemplary air purification system.

FIG. 3 is a flow chart showing operations in a process for producing bacterial biomass and purifying air.

### DETAILED DESCRIPTION

This disclosure describes a reaction system that includes a photochemical fiber reactor to convert CO₂ and other atmospheric components into food (or biomass) using bacteria harvested from the air. The system can include a single reaction chamber that operates with air as a feedstock. Air containing carbon (e.g., in the form of CO₂), atmospheric particulates including nitrogen (e.g., in the form of ammonium nitrate (NH₄NO₃), ammonium sulfate ((NH₄)₂SO₄), reactive nitrogen species (NOY) including nitrogen oxides (NO_{X})), phosphorous (e.g., volcanic ash, sea salt, phosphine (PH₃), soil particles, polyphosphates from industrial emissions), and bacteria is delivered to the reactor. The reaction chamber can include water as the reaction medium. Air containing sources of carbon, nitrogen, and bacteria is wet scrubbed through the water in the reaction medium to capture the carbon, nitrogen, and bacteria. Other capture methods include passing air through suitable air filters. The carbon, nitrogen, and bacteria are wet scrubbed into the reaction medium, creating a bacterial growth solution containing atmospheric components, essential trace nutrients (e.g., sources of nitrogen, sources of phosphorus), or a combination of both. Examples of suitable bacteria include strains such as Methylobacteria, Azotobacter, Cyanobacteria, Acetobacter, and Nitrosomonas bacteria.

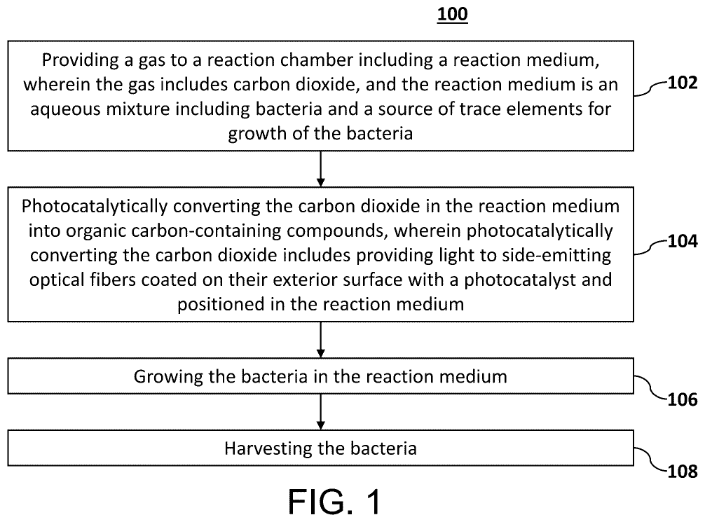

FIG. 1 is a flow chart showing operations in a process 100 for producing biomass. In 102, a gas is provided to a reaction chamber including a reaction medium. The gas typically includes carbon dioxide. A suitable example of the gas is air. The reaction medium is typically an aqueous mixture including bacteria and a source of trace elements for growth of the bacteria. The reaction medium can be atmospheric water (e.g., collected from fog or clouds). Suitable examples of trace elements include nitrogen and phosphorous (e.g., in the form of phosphine (PH₃), phosphates, polyphosphates, phosphoric acid (H₃PO₄), particulate organic phosphorus). In some cases, the air includes trace elements. The gas typically includes particulate matter, and the particulate matter can be the source of the trace elements. A suitable example of particulate matter includes dust. The source of the trace elements for biological growth typically includes dust, aerosols, or other particulates from the air. In some cases, the process 100 further includes providing the dust, aerosols, or other particulates to the reaction chamber. Providing the dust to the reaction chamber typically includes filtering the dust, aerosols, or other particulates from air. In 104, the carbon dioxide is photocatalytically converted in the reaction medium into organic carbon-containing compounds (e.g., formaldehyde and formic acid). Photocatalytically converting the carbon dioxide typically includes providing light to side-emitting optical fibers on their exterior surface with a photocatalyst and positioned in the reaction medium. In 106, the bacteria are grown in the reaction medium. In 108, the bacteria are harvested. The bacteria typically includes Proteobacteria, uncultured Caulobacteraceae, Bacteroidota, Methylobacteria, Massilia, Sphingomonas, Bacteroidetes, Firmicutes, Actinobacteria, Cyanobacteria, or a combination thereof.

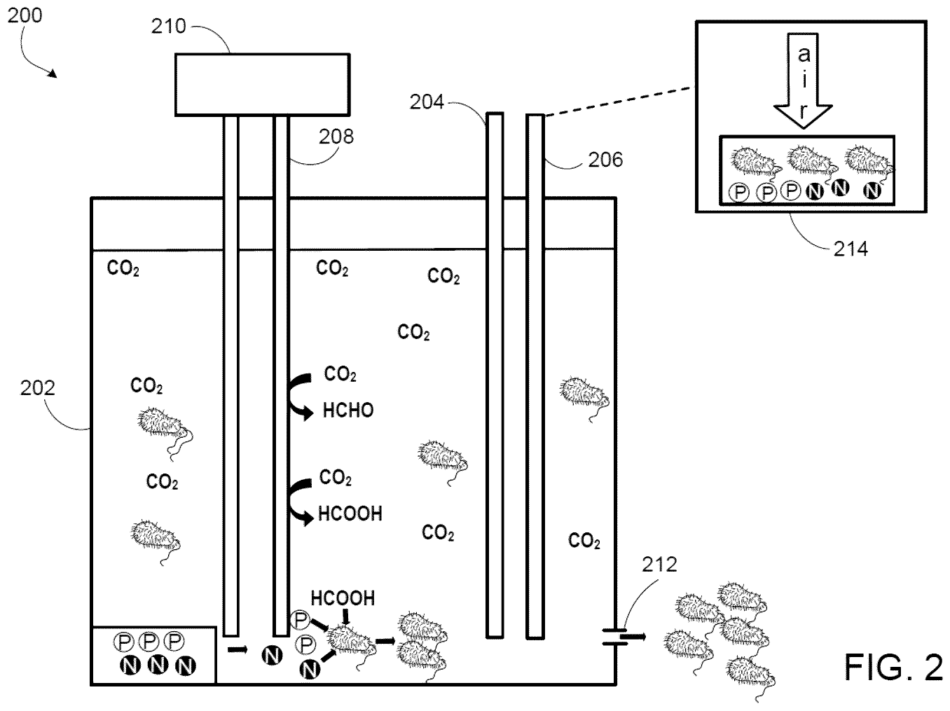

FIG. 2 shows an example of an air purification system 200 with a single reaction chamber 202 that uses sunlight, atmospheric gases, atmospheric particulates, and bacteria from atmospheric dust to produce food or biomass. The reaction chamber 202 is supplied with water as a reaction medium. Atmospheric water (e.g., from fog or clouds) can be harvested to provide the water used in the reaction chamber 202. Atmospheric water vapor can be collected by condensing upon contact with a cold surface and collecting the condensed liquid water. Other methods to condense water from the air include using compressor or desiccant-based systems. Atmospheric water can be collected from passive collection systems such as dew nets or fog catchers. Water can be supplied from fresh water sources including groundwater, rainwater, rivers, lakes, streams, and the like. Water can also be supplied from tap water, or non-fresh water sources including brackish water, seawater, recycled water, stormwater, or other available water sources. The use of non-fresh water sources alleviates the strain on freshwater resources.

A first feedstock including CO₂ can be supplied to the reaction chamber 202 through a first inlet 204. The CO₂ can come directly from ambient air or be provided by an external, concentrated source. Examples of concentrated CO₂ sources include CO₂ emitted from flue gas stacks from sources such as fossil fuel and natural gas plants. Additional components can be included in the CO₂ source, and the CO₂ does not need to be purified before use. In some cases, the first feedstock is air, and the first inlet 204 is configured to accept the air and provide the air to an aqueous reaction medium in the reaction chamber 202.

A second feedstock including particulate matter from ambient air can be supplied to the reaction chamber 202 through a second inlet 206. In some implementations, the first inlet 204 and the second inlet 206 are the same, the first feedstock and the second feedstock are the same, and the first feedstock and the second feedstock is air.

The reaction chamber 202 also includes one or more side-emitting optical fibers 208. The one or more side-emitting optical fibers 208 typically include a photocatalytic exterior coating. The one or more side-emitting optical fibers 208 can be configured to be optically coupled to a light source 210. Light can be guided through the optical fibers 208 into the reaction chamber 202. Light can interact with the photocatalyst coated on the outer surface of the optical fibers 208 and CO₂ in the reaction chamber 202 in a photochemical reaction to convert CO₂ to organic one-carbon compounds (e.g., formaldehyde, formic acid, methanol). Sunlight can be used during daytime hours. Light produced from electricity (e.g., light emitting diodes) can be used anytime, and at wavelengths optimized to activate the photocatalysts. An outlet 212 can be configured to allow harvesting of the bacteria from the single reaction chamber 202.

In some cases, the air purification system 200 includes a system 214 configured to capture particulate matter from ambient air and provide the particulate matter to the reaction chamber 202. The system 214 is typically configured to be operatively coupled to the single reaction chamber 202. The system 214 can include a filter. The filter 214 can be configured to capture particulate matter from ambient air.

Ambient air can be filtered to collect airborne components such as atmospheric dust, particulates, and bacteria. The collected airborne components can be passed into water (e.g., wet scrubbing) located in the reaction chamber 202. Atmospheric dust can include soil dust, sea spray, volcanic ash, or bioaerosols such as plant debris, animal debris, bacteria, and viruses. Atmospheric particulates can include sources of nitrogen and phosphorus. Nitrogen-containing atmospheric particulates typically include ammonium nitrate, ammonium sulfate, and gaseous nitrogen containing species reacting with water and condensing on atmospheric dust. Phosphorus-containing atmospheric particulates typically include phosphate minerals, combustion emissions, mineral dust, primary biological aerosol particles, sea salt, volcanic ash, plant debris, microorganisms, and phosphine (e.g., from wetlands). Types of bacteria in the atmosphere can include Proteobacteria, uncultured Caulobacteraceae, Bacteroidota, Methylobacteria, Massilia, Sphingomonas, Bacteroidetes, Firmicutes, Actinobacteria, and Cyanobacteria. In some cases, nitrogen, phosphorus, and suitable bacteria are provided from external inputs, such as laboratory-grade chemicals, commercially available microbial cultures, or industrial emissions.

The bacteria in the reaction chamber 202 metabolize C₁ and longer-chain carbon compounds generated from CO₂ supplied to the reaction chamber 202 and converted by the photocatalytically coated optical fibers 208. The bacteria in the reaction chamber 202 can also metabolize sources of nitrogen and phosphorus. The bacteria can use the C₁ and longer-chain carbon compounds and sources of nitrogen and phosphorus for energy needs and growth, creating an increase in biomass. The generated biomass can be recovered and transformed into food or value-added materials. In one example, the biomass is approximately 50% protein and can include lipids and carotenoids. In one example, the biomass is approximately about 40% to about 80% protein. Converting bacteria to food addresses food scarcity by producing biomass using components derived from the atmosphere that can serve as a food source.

Some bacteria convert light energy from the sun into chemical energy by absorbing CO₂ and H₂O (e.g., biological photosynthesis), though most use organics and an oxidant to produce CO₂ and H₂O. Bacteria can grow and reproduce over a range of conditions (e.g., pH, temperature, salinity) to increase cellular biomass without the need for biological photosynthesis. In the reaction chamber 202, light (e.g., sunlight) delivered into the photocatalyst coated optical fibers 208 converts CO₂ into organic C₁ or longer carbon chain compounds that are used as an energy and carbon source for the bacteria. The light source 210 can be an artificial light source or natural sunlight concentrated using lenses or mirrors and then supplied to the reaction chamber. The light can be filtered to limit the growth of undesirable phototrophic organisms while still allowing for the photolytic conversion of CO₂ into C₁ compounds.

The reaction chamber 202 can use externally provided, non-ambient atmospheric sources of water, energy, nitrogen and phosphorous while using microorganisms present in the atmosphere. In some cases, the reaction chamber 202 is used as an air purification system by removing dust, particulates, atmospheric gases such as CO₂, nitrogen oxides and C₁ pollutants such as formaldehyde from the ambient air (e.g., wet scrubbing). In some cases, the reaction chamber 202 operates independently, functioning without the need for external input sources other than ambient air. The reaction chamber 202 can be integrated with carbon capture devices or industrial exhaust streams to assist in controlling emissions. The reaction chamber 202 can operate using ambient air as an input source. The reaction chamber 202 can operate using water from wastewater sources (e.g., wastewater treatment plants). These environments can provide sources of anaerobic bacteria and organic matter for conversion to biomass.

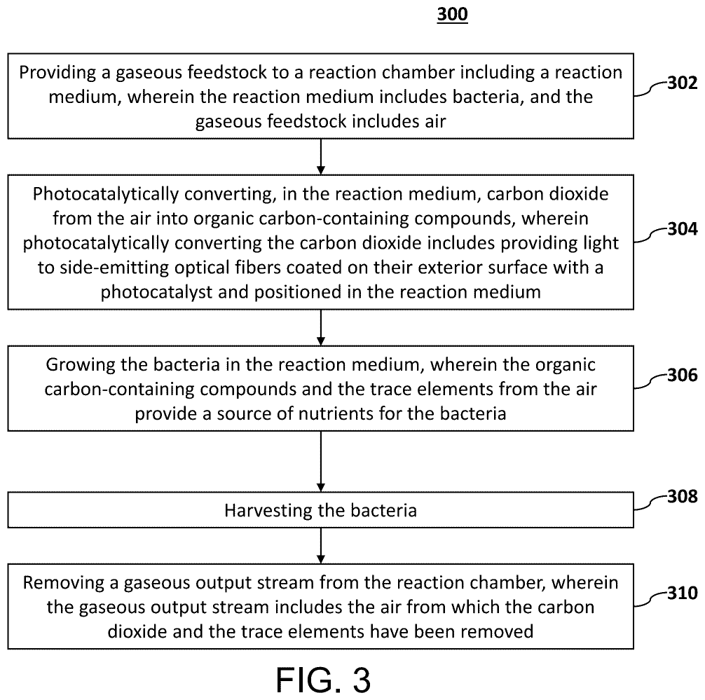

FIG. 3 is a flow chart showing operations in a process 300 for producing bacterial biomass and purifying air. In 302, a gaseous feedstock is provided to a reaction chamber including a reaction medium, where the reaction medium includes bacteria, and the gaseous feedstock includes air. In some cases, the process 300 further includes removing particulate matter from the air prior to providing the gaseous feedstock to the reaction chamber. The bacteria can be provided to the reaction chamber via particulate matter from the air. In 304, in the reaction medium, carbon dioxide from the air, is photocatalytically converted into organic carbon-containing compounds. Photocatalytically converting the carbon dioxide includes providing light to side-emitting optical fibers coated on their exterior surface with a photocatalyst and positioned in the reaction medium. In 306, the bacteria are grown in the reaction medium, where the organic carbon-containing compounds and the trace elements from the air provide a source of nutrients for the bacteria. In 308, the bacteria are harvested. In 310, a gaseous output stream is removed from the reaction chamber. The gaseous output stream typically includes the air from which the carbon dioxide and the trace elements have been removed. In some cases, the gaseous output stream includes the air from which the particulate matter has been removed. The process 300 can further include processing the bacteria to yield foodstuff.

### EXAMPLES

### Example 1. Radiation Fog Sample Collection and Analyses

To assess if atmospheric water droplets can function as microbial habitats, a collection of samples in 32 radiation fog events over central Pennsylvania spanning two years was conducted. Unlike advection fogs or clouds, radiation fogs form in localized areas, typically in stagnant air masses, providing an opportunity to detect differences between activated droplets and inactivated aerosol particles bacteria in time and space. Bacterial composition and the potential dynamic influence of fog formation on microbial partition between fog droplets and interstitial aerosol particles in fog, growth potential, and microbial metabolic activity were determined.

Fog and aerosol samples were collected at Susquehanna University's Center for Environmental Education and Research (CEER; 40.79° N 76.88° W) near Selinsgrove, PA. The site was characterized by open grassy fields and surrounded by active farmland. Fog samples were collected with an automated Caltech Heated Rod Cloudwater Collector (CHRCC) having a 50% cut-off particle diameter of approximately 9 μm mounted 2 m above ground. The rods were not heated. The CHRCC draws air at 5.8 m³ min⁻¹ over a bank of 3.2 mm diameter stainless steel impaction rods where fog droplets accumulate and then flow into a sterile polyethylene bottle. A single sample was collected during each fog event. At least in part because radiation fog formation can be difficult to predict, the operation of the CHRCC was automated to promote reliable sample collection. A Belfort model 3100 visibility monitor was used to detect the presence of fog and to start collection by opening pneumatic doors at the CHRCC's inlet and outlet and activating the CHRCC's fan. A visibility threshold of 0.5 km was used to indicate the presence of dense fog. When visibility remained below 0.5 km for at least five minutes, collection was initiated and then continued until visibility exceeded this threshold for five minutes. The collector was washed with 1 L of distilled water in the evenings before fog was expected to form. After washing, an additional 100 ml of distilled water was sprayed onto the collection surfaces and used as a blank. After fog dissipation, samples were transported for analysis and kept at 4° C. for up to three weeks before anion analysis. Whenever the CHRCC was activated, an interstitial aerosol particles sample was simultaneously obtained, by collection on 47 mm quartz fiber filters (Whatman QM-A, Cytiva, MA, USA; prebaked at 600° C. for 12 hours). These filters were housed in an Advantec MFS polypropylene, open-faced filter pack installed downstream of the CHRCC exit. A shroud was constructed around the filter pack to allow only air and particles that had passed through the CHRCC to be sampled. The flow rate through the filter pack was 22 L min⁻¹ controlled by a critical orifice. In addition to the interstitial aerosol particles samples, ambient aerosol samples were obtained before and after fog formation. The sample duration for the pre- and post-fog aerosol samples was 12 hours, using the same quartz fiber filters and flowrate as for interstitial samples. The filter packs were housed underneath a rain shield located about 2 m from the CHRCC.

Concentrations of inorganic anions in the fog samples and blanks were determined by ion exchange chromatography using a Thermo Scientific Dionex Integrion system with suppressed conductivity detection. Separation was obtained with a Dionex AS18 column and KOH eluent at an isocratic flow rate of 1 ml min⁻¹.

The collection rate of each fog sample Cr (g min⁻¹) was calculated based on collected sample volume (g) and sampling time (min). The liquid water content (LWC) (g m⁻³) was calculated by applying the equations 1 and 2:

$$LWC = - 0.092 {C}_{r}^{2} + 0.31 Cr + 0. 0 \; 023 for 0.025 \leq LWC \leq 0.175 \qquad ( 1 )$$

$$LWC = 0.235 Cr + 0.0 \; 51 for LWC > 0.175 \qquad ( 2 )$$

The air volume fraction of fog droplets vi (vol %) was calculated by dividing the LWC (mg m⁻³) by the water density (ρ=106 mg m⁻³).

To assess potential bacterial growth in situ, the frequency of dividing cells (FDC) was determined through microscopic counts on formaline-fixed samples. 15 ml of fog sample was fixed with a 37% formaldehyde solution (Sigma-Aldrich, MO, USA) to a final concentration of 4% and kept at 4° C. Bacteria in the samples were stained by addition of DAPI (4′,6-diamidino-2-phenylindole, 0.03M, Sigma-Aldrich, MO, USA) for 2 minutes, and collected onto black 0.2 μm pore-size polycarbonate membrane filters (Sterlitech, WA, USA) by filtration. The filters were placed on microscope slides for inspection using epifluorescence microscopy. For interstitial aerosol samples, half of the collecting quartz filter was cut and soaked in 4% formaldehyde solution for 10 minutes. The filter was then dried and stored at 4° C. The bacteria on the filter were resuspended in 10 ml of phosphate buffer saline (PBS, pH=7.0), stained with DAPI and collected on polycarbonate filters as with the fog samples. The cells were visualized with epifluorescence microscopy (Carl Zeiss AxioScope.A1) using ultraviolet excitation (380 nm-400 nm). FDC was calculated as the number of dividing cells over the total cell count under the microscope. A cell was considered dividing when a cell wall constriction was visible. For each determination, 1,500 cells in at least 10 microscopic fields were counted. The length and width of DAPI-stained cells observed under the microscope were measured using ImageJ software version 1.54. Cell volume was derived from measuring dimensions assuming simple formulae for cylinders. One thousand cells each were counted to generate the cell volume distribution of each sample type.

To determine particle size distributions through flow cytometry, one half of each filter (quartz or polyethersulfone) was resuspended in 10 ml of phosphate buffer saline at 4° C. for 12 hours. The solution was injected into an Attune NxT flow cytometer (Thermo Fisher Scientific, MA, USA) to measure particle size distribution using the forward scatter detector with a blue (488 nm) laser light source. A series of artificial 2.00, 3.30, 5.17, 7.56, 10.1, and 16.5 μm diameter polystyrene particles (Spherotech, IL, USA) at a concentration of 10⁵ ml⁻¹ were used as size standards. Size range gates were generated by matching gate extrema to the median intensity of the forward scatter signal for each bead size. Percentage of total events whose intensities fall into each discrete size range was determined using the FlowJo™ platform version 10.

Samples were processed immediately after collection. 20 ml to 100 ml of liquid fog sample was filtered through a sterile 47 mm filter funnel provided with a polyethersulfone membrane filter with a 0.2 μm diameter pore size (Pall, NY, USA), then kept at −20° C. until further processing. Deoxyribonucleic acid (DNA) extraction used the PowerSoil DNA Extraction Kit (MoBio/QIAGEN, Germany) with several modifications. Half of the polyethersulfone filter from the fog sample was used for DNA extraction and was cut into 0.25 cm² pieces with a sterile surgical scalpel. Pieces were then rolled with sterile forceps and inserted into three PowerBead Pro Tubes with 800 μl of CDI solution, 250 μl of phosphate buffer saline, and 150 μl of phenol:chloroform:isoamyl solution (25:24:1). The mixture was homogenized using a Mini-Beadbeater-16 (Biospec) with intervals of 30 seconds of bead-beating and cooling on ice until filters were shredded. DNA extraction followed the manufacturer's protocol, and three extracts were combined in one MB Spin Column at the DNA binding step. After washing the column per protocol, 500 μl of 100% ice-cold ethanol was used to remove residues. Finally, DNA was eluted and stored at −20° C. until downstream analysis. This same protocol was used in the quartz fiber filter from aerosol samples. This protocol was also used to detect potentially contaminant DNA from handling by assessing the blank samples. The blank samples included the distilled water used to wash the fog sampling devices.

Ribosomal RNA genes were amplified and barcoded by polymerase chain reaction (PCR) from DNA extracts using primers 515F/806R targeting the V4 region of the 16S rRNA gene. PCR products were pooled, purified, and mixed for MiSeq sequencing following the reference method. High-throughput sequencing was performed on the Illumina platform with the MiSeq instrument by Microbiome Analysis Laboratory at Arizona State University (AZ, USA), yielding raw FASTQ sequence files. The obtained sequences were analyzed via QIIME2 (v2021.11) using the DADA2 plugin to create a feature table containing representative sequences (amplicon sequence variants (ASVs)) and their frequencies of occurrence. The lowest number of sequences in one sample was 3,450, and the highest was 152,049 reads. The taxonomic assignments were then aligned against the SILVA database core reference alignment. For further analysis sequences assigned to bacteria were kept, but not those assigned to eukaryotic organelles (chloroplasts or mitochondria). Then, the relative abundance of a bacterial taxon was defined as the number of reads assigned to the taxon divided by the total number of reads per sample. To conduct comparative alpha and beta diversity analyses using a common denominator of sampling effort, the reads of each sample were trimmed to 4,942, keeping most samples (one that had only 3,450 reads was discarded), while maintaining a good level of diversity saturation (90% of Shannon Diversity saturation), and without relevant effects on community diversity assessment. For the subset of 6 fog events with paired determinations of all types (pre-fog aerosol, fog droplets, interstitial aerosol particles in fog, and post-fog aerosol), samples were trimmed at the depth of the lowest sample (4,638 reads) for the diversity analysis. Alpha and beta diversity metrics were computed using the diversity plugin available on QIIME2. Principal Coordinates Analysis (PCoA) was used for the analysis of the Bray-Curtis and Jaccard distance matrix from QIIME2 (plotted on the R vegan package). To determine which ASVs were differentially abundant in fog and interstitial aerosol particles communities, the DeSeq2 method was used on the R BiocManager package. Any ASVs detected as different that were also detected in the blank wash water were discarded as potential contaminants, but this was only done in a few cases.

DNA concentrations in the extracts were determined via fluorometry using Qubit™ dsDNA high sensitivity Assay Kits (Life Technologies, CA, USA). The copy numbers of 16S rRNA genes in extracts were quantified by real-time quantitative polymerase chain reaction (qPCR), using the universal primers 338F 5′-ACTCCTACGGGAGGCAGCAG-3′ and 518R 5′-GTATTACCGCGGCTGCTGG-3′, performed in triplicate using PerfeCTa SYBR® Green FastMix Rox (Quantabio, MA, USA) in an ABI ViiA 7 thermocycler (Applied Biosystems, MA, USA). The calculated 16S rRNA gene copy number in each sample was multiplied by the percentage of filtered bacterial reads to attain corrected prokaryote 16S rRNA gene copy number. The air-equivalent concentration of bacteria was defined as the 16S rRNA gene copy number obtained per unit volume of air sampled (copies m⁻³). For aerosol particles, this concentration was calculated from the qPCR data (copies per extract) and the total air volume sampled. For the fog samples, the air-equivalent concentration is equal to the cell liquid concentration (copies ml⁻¹) multiplied by the liquid water content (LWC, mg m⁻³) and assuming that water density ρ=1 g ml⁻¹.

Fog water collected by CASCC during 4 different events was transferred immediately to the lab for biodegradation test. A killed control was prepared by adding chloroform (Sigma-Aldrich, MO, USA) at a 1:30 ratio to the fog water. A particle free control was also prepared by filtration through a polyethersulfone filter package with nominal porosity of 0.22 μm (Pall, NY, USA) to remove microorganisms. Native fog water and controls were incubated in brown, 250-mL glass bottles at 10° C. (average temperature of the fog events in the late summer and fall) in the dark conditions for 5 days. Aliquots were collected at the beginning of the experiment and at a 12-hour interval, preserved with a sulfur (IV) formaldehyde preservative solution and kept at 4° C. before formaldehyde determination by fluorometry. The biotransformation rates (M s⁻¹) were calculated following the first order loss rate. The cell-specific rates (mol cell⁻¹ s⁻¹) were derived by dividing the biotransformation rate to the prokaryote 16S rRNA gene copy number (copies ml⁻¹).

All the statistical analyses were performed on the R vegan package. Levene's test was used to test for equal variance and Shapiro-Wilk Test for normality. After verifying the assumptions met, Analysis of Variance (ANOVA) statistical test was applied, and Tukey's honest significant difference (HSD) was used as a post-hoc test for pairwise comparisons. The Wilcoxon test was used for pairwise comparisons of datasets failing normality tests.

The fog water microbiome was gauged by the number of bacterial copies of the 16S rRNA gene, 8.3±7.7×10⁵ copies per ml of liquid, but also variable among events. Biological loads assessed by total DNA concentration gave consistent results, indicating that bacteria were determinant of the total biological load in fog water. Considering the habitat where bacteria are present (e.g., fog droplet vs. aerosol particle), air-equivalent concentrations of bacteria in a fog-free acrobiome at the site was smaller (pre-fog and post-fog conditions, n=11), averaging 0.1 bacterial 16S rRNA gene copies of bacteria per ml of air, between 6 and 7 orders of magnitude more dilute than 1.1±0.8×10⁶ copies per ml of fog water. This concentration effect was assessed in a subset of six events, where concurrent fog droplets and interstitial aerosol particles during fog were sampled. The number of bacterial copies suggested that fog droplets represent a major bacterial hub in the air during fog events. Aqueous bacterial concentrations in fog droplets tended to increase with the fog LWC, though a regression gave R²=0.02, p=0.49 for the full data set, and R²=0.14, p=0.09 when removing two outliers. This was in comparison to the lack of trends between aqueous ionic content and LWC (a power regression yielded R²=0.12, p=0.07). The ratio of biological vs. chemical contents in fog was proportional to its LWC (R²=0.14, p=0.05, n=28, and R²=0.27, p=0.006 excluding two outliers). This suggested a different processes control concentrations of bacteria vs. solutes in fog droplets. Fog water microbiome size tended to increase with increasing ambient temperatures, the square root of bacterial concentration being linearly related to temperature (R²=0.36, p=3×10⁻⁴ for the full data set), following the relationship of bacterial growth rate with temperature. These two relationships were consistent with in situ growth playing a role in the concentration of the fog water microbiome. And yet, bacterial numbers seemed independent of event duration (an exponential regression gave R²=0.02, p=0.43 even with two outliers excluded). Bacterial concentrations reached during short events (duration <2 h) could be as high as those in long events. Hence, while bacterial growth can be partly responsible for the size of the fog microbiome, factors that are virtually instantaneous at the scale of hours also contributed to concentrating bacteria from aerosols into droplets immediately upon fog condensation.

Upon 16S rRNA gene sequencing of fog water samples, an average of 188 unique ASVs per event were detected, though this was also quite variable (standard deviation=106 ASVs). At the phylum level, Proteobacteria tended to dominate most of the fog microbiomes with 65±18% of reads, followed by Firmicutes (12±12%) and Bacteroidota (7±11%). At the genus level, bacteria in the Methylobacterium-Methylorubrum group were consistently dominant, contributing 29±13% of all reads, followed by Massilia (9±20%), uncultured Caulobacteraceae (6±5%), and Sphingomonas (5±4%). The representation of those secondary bacterial taxa was less consistent than that of Methylobacterium-like phylotypes. Methylobacterium bacteria are heterotrophs that specialize in C₁ compounds, except methane, through the serine pathway of C₁ oxidation. A viable count in agar media containing 100 mM formaldehyde as the sole carbon source yielded 2620 colony-forming units per ml of fog water, in which all colonies had the pink phenotype typical of Methylobacter.

The dynamics of the acrobiome during fog formation and dissipation with respect to population size during the 6 events was determined. The biological content in aerosols (pre-fog, interstitial during fog, and post-fog) as well as fog water in each event was computed on a common volumetric basis. At least in part because fog droplets occupy a small fraction (1.3×10⁻⁵ percent) of the total air volume, the total population sizes of droplet and interstitial microbiomes within a given volume of air tended to be commensurate (n=6, T-test, p=0.76). During fog, bacterial concentrations in the air (combined dry and wet fractions) were higher than in the pre-fog air (Wilcoxon signed-rank p=0.03), increasing anywhere from 1.5-fold to 16-fold. The calculated LWC from the collection rate can vary by 20% and influence the air-equivalent bacteria concentration in fog droplets by the same relative amount. Using total air-equivalent concentrations of bacteria during the fog event and pre-fog aerosol particles, the calculated microbiome generation rates were 0.67±0.64 h⁻¹, consistent with well-known bacterial strains across various ecosystems. However, this is challenging to account for without invoking net local growth, particularly in the absence of allochthonous inputs under air stagnation. After dissipation, the number of bacteria dropped again (p=0.08, Wilcoxon signed-rank), although the post-fog aerosol particles retained a 115 higher biological content than the respective pre-fog air (Wilcoxon signed-rank p=0.02), if only by 45% on average. These general dynamics speak for a transient burst in the acrobiome population size during a fog event, followed by an incomplete but significant contraction upon fog dissipation. The loss factors responsible for this contraction can be either death caused by desiccation at the end of the event, or export (e.g., wet deposition) during the event. The loss ratio across a fog event (number of bacteria after vs. before) was inversely proportional to its duration, which suggested that losses occurred largely through a continuous process of deposition. This can also explain why an increase of the fog microbiome with event duration was not observed, as both processes (growth and loss) would typically cancel each other.

To further probe the possibility of active growth in the fog water, the bacterial populations was examined directly by epifluorescence microscopy and flow cytometry. Anything collected by the fog collector was considered as fog water and anything collected on the aerosol filter downstream of the fog collector was considered as interstitial aerosol. This differentiation may not be accurate at least in part because of the non-ideal collection efficiency curves inherent to inertial collectors, such as the CHRCC, and the possibility that larger organisms (e.g., >9 μm) that are not activated can be collected by the CHRCC. However, the results indicated that such organisms are rare. The size distribution showed that the bacteria present in fog droplets were larger than those in aerosol form (8.5±1.2 vs. 1.6±3.8 μm³; Wilcoxon rank-sum test, p<2×10⁻¹⁶), though both were well within the range encompassing the majority of bacterial species. This was consistent with active growth in the fog water, since growing cells attain larger volumes than their non-growing counterparts. The same trend was observed independently through automated particle sizing by flow cytometry. FDC determinations were also used to assess in situ growth. The FDC in fog water samples ranged from 1.5% to 3.5%, with an average of 2.4%, which in aquatic microbiomes corresponds to doubling times of some 60 hours. In interstitial aerosol particles during fogs, the range was from 0.5 to 1.4%, with an average of 1.0%. The two frequencies were different according to a Wilcoxon rank-sum test for paired values on angular-transformed data with a p=0.03.

In the 6 fog events with paired determinations of all microbiome types, indices of microbial diversity were similar among them, but, in spite of the variations in composition among fog events, fog water microbiomes tended to be self-similar, and differentiated from the microbiomes existing in the air prior to fog condensation. They also differed in composition from the interstitial (e.g., aerosol phase) microbiomes during fog events themselves. The Bray-Curtis dissimilarity algorithms used factor in differences in relative abundance, but those using Jaccard distances did not. That communities could no longer be distinguished from one another in the latter indicated that fog water microbiomes are likely sourced in the aerosol microbiome, differentiating by preferential enrichment of particular types, rather than by incorporation of new types. Fog water microbiomes were also compositionally different from those in fog-free air after a fog event came to an end, although less so than from those present in the aerosol phase prior or during the fog event itself. The selectivity in partitioning appeared to leave a weak imprint on the acrobiome composition after fog dissipation. A comparison of microbial composition in the subset of paired concurrent fog water/aerosol samples can be used to discern which microorganisms were preferentially enriched in each compartment. Preference for water droplets involved ASVs assignable to Methylobacterium, the Caulobacteraceae or the Beijerinckaceac, whereas others like Bradyrhizobium appeared to be preferentially found as dry aerosols. DESeq2 analyses on cumulative data from the 6 events showed that only 7 among 1320 ASVs found were differentially enriched (p<0.05) in droplets. Among those, the Methylobacterium-Methylorubrum ASVs were by far the most abundant (14 to 50% of total reads in fog water, but only up to 0.3% in interstitial aerosol particles). No ASVs reached preferential enrichment in aerosols.

Considering fog as a habitat brought forth the question of what type of metabolism supports it. A trait-based analysis of the ASVs preferentially enriched in fog water offered guidance. Since none could be assigned to known phototrophs, the basis was most likely chemotrophy. No phylotypes fit known chemolithotrophs, supporting that the system was most likely a chemoorganotrophic system. As a source of energy and carbon, fog bacteria can principally count on an ample supply of volatile organic compounds (e.g., alcohols, aldehydes, and organic acids). The recurrent dominance of Methylobacterium suggested that the fog microbiome could be based on the conversion of volatile C₁-type organics. The removal of formaldehyde, a common (and toxic) volatile in the atmosphere, through incubations of freshly collected fog water was assessed. Formaldehyde (present at concentrations 6 μM-25 μM) was consumed to undetectable levels during incubations, with rates around 2.9±1.8×10⁻¹⁰ M s⁻¹ (or some 25 μM day⁻¹), roughly two-hundred-fold faster than those measured in cloud water. The incubations were conducted in a closed system that could have constrained for substrate replenishment from the gas phase, limiting available formaldehyde for consumption. On the other hand, at least in part because bacteria are not present in all droplets and the chemical composition of droplets is known to be heterogeneous, bacteria might have been physically separated from essential formaldehyde in a given droplet. This effect can overestimate the conversion rate. These two effects can alter the measured in situ metabolic rate from the metabolic rate of the actual atmospheric setting. On a per cell basis, derived using microscopic cell counts in the incubation water, this corresponds to 2.3±1.4×10⁻¹⁸ mol cell⁻¹ s⁻¹, 30-fold faster than when measured in cloud water, and close to the maximal rates measured in the lab for pure cultures of bacteria. The activity is associated largely with the particulate fraction in the fog water samples, as it could be virtually halted by filtration, and the controls using chloroform-killed fog water showed that the majority of formaldehyde transformation (95% on average) in the fog samples could be ascribed to biological activity. If the extant bacterial population were oxidizing formaldehyde solely to support growth with growth efficiencies typical of methylotrophs that use the serine pathway in culture [around 8 g (dry mass) per mol of substrate], they could theoretically be doubling biomass in a few minutes. This is an unlikely rate of biomass production for known microbes. Biological formaldehyde degradation likely served energy generation or mere detoxification purposes, rather than exclusively support growth.

Although this disclosure contains many specific embodiment details, these should not be construed as limitations on the scope of the subject matter or on the scope of what may be claimed, but rather as descriptions of features that may be specific to particular embodiments. Certain features that are described in this disclosure in the context of separate embodiments can also be implemented, in combination, in a single embodiment. Conversely, various features that are described in the context of a single embodiment can also be implemented in multiple embodiments, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular embodiments of the subject matter have been described. Other embodiments, alterations, and permutations of the described embodiments are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Accordingly, the previously described example embodiments do not define or constrain this disclosure. Other changes, substitutions, and alterations are also possible without departing from the spirit and scope of this disclosure.

What is claimed is:

1. A method for producing biomass, the method comprising:
providing a gas to a reaction chamber comprising a reaction medium, wherein the gas comprises carbon dioxide and the reaction medium is an aqueous mixture comprising bacteria and a source of trace elements for growth of the bacteria;
photocatalytically converting the carbon dioxide in the reaction medium into organic carbon-containing compounds, wherein photocatalytically converting the carbon dioxide comprises providing light to side-emitting optical fibers coated on their exterior surface with a photocatalyst and positioned in the reaction medium;
growing the bacteria in the reaction medium; and
harvesting the bacteria.

2. The method of claim 1, wherein the gas comprises air.

3. The method of claim 1, wherein the trace elements comprise nitrogen and phosphorus.

4. The method of claim 2, wherein the air comprises the trace elements.

5. The method of claim 1, wherein the gas comprises particulate matter, and the particulate matter is the source of the trace elements.

6. The method of claim 5, wherein the particulate matter comprises dust.

7. The method of claim 1, wherein the source of the trace elements essential for biological growth comprises dust, aerosols, or other particulates from the air.

8. The method of claim 7, further comprising providing the dust, aerosols, or other particulates to the reaction chamber.

9. The method of claim 8, wherein providing the dust to the reaction chamber comprises filtering the dust, aerosols, or other particulates from air.

10. The method of claim 1, wherein the bacteria comprise Proteobacteria, uncultured Caulobacteraceae, Bacteroidota, Methylobacteria, Massilia, Sphingomonas, Bacteroidetes, Firmicutes, Actinobacteria, Cyanobacteria, or a combination thereof.

11. An air purification system comprising:
a single reaction chamber configured to contain bacteria;
a first inlet configured to accept a first feedstock comprising carbon dioxide;
a second inlet configured to accept a second feedstock comprising particulate matter from ambient air;
one or more side-emitting optical fibers configured to be positioned in the single reaction chamber, wherein the one or more side-emitting optical fibers comprise a photocatalytic exterior coating and are configured to be optically coupled to a light source and convert the carbon dioxide in the single reaction chamber to organic carbon compounds; and
an outlet configured to allow harvesting of the bacteria from the single reaction chamber.

12. The air purification system of claim 11, wherein the first feedstock is air, and the first inlet is configured to accept the air and provide the air to an aqueous reaction medium in the single reaction chamber.

13. The air purification system of claim 11, wherein the first inlet and the second inlet are the same, the first feedstock and the second feedstock are the same, and the first feedstock and the second feedstock is air.

14. The air purification system of claim 11, further comprising a system configured to capture particulate matter from ambient air and provide the particulate matter to the single reaction chamber.

15. The air purification system of claim 14, wherein the system is configured to be operatively coupled to the single reaction chamber.

16. The air purification system of claim 14, wherein the system comprises a filter.

17. The air purification system of claim 16, wherein the filter is configured to capture the particulate matter from the ambient air.

18. A method for producing bacterial biomass and purifying air, the method comprising:
providing a gaseous feedstock to a reaction chamber comprising a reaction medium, wherein the reaction medium comprises bacteria, and the gaseous feedstock comprises air;
photocatalytically converting, in the reaction medium, carbon dioxide from the air into organic carbon-containing compounds, wherein photocatalytically converting the carbon dioxide comprises providing light to side-emitting optical fibers coated on their exterior surface with a photocatalyst and positioned in the reaction medium;
growing the bacteria in the reaction medium, wherein the organic carbon-containing compounds and trace elements from the air provide a source of nutrients for the bacteria;
harvesting the bacteria; and
removing a gaseous output stream from the reaction chamber, wherein the gaseous output stream comprises the air from which the carbon dioxide and the trace elements have been removed.

19. The method of claim 18, further comprising processing the bacteria to yield foodstuff.

20. The method of claim 18, further comprising removing particulate matter from the air prior to providing the gaseous feedstock to the reaction chamber.

21. The method of claim 18, wherein the bacteria are provided to the reaction chamber via particulate matter in the air.

22. The method of claim 21, wherein the gaseous output stream comprises the air from which the particulate matter has been removed.
